(19) United States
(12) Patent Application Publication
(10) Pub. No.: US 2014/0180186 A1
(43) Pub. Date: Jun. 26, 2014

(54) POSTURE-DEPENDENT ACTIVE TORSO SUPPORT

(71) Applicant: ELWHA LLC, Bellevue, WA (US)

(72) Inventors: Roderick A. Hyde, Redmond, WA (US); Jordin T. Kare, Seattle, WA (US); Dennis J. Rivet, Chesapeake, VA (US); Lowell L. Wood, JR., Bellevue, WA (US)

(73) Assignee: ELWHA LLC, Bellevue, WA (US)

(21) Appl. No.: 13/721,474

(22) Filed: Dec. 20, 2012

(51) Int. Cl. A61F 5/02

(52) U.S. Cl. CPC A61F 5/02
USPC 602/19

(57) ABSTRACT

An active torso support that controls force applied to one or more portions of a torso of a subject in response to detected posture of the subject is described. The active torso support includes one or more elements for applying force to the torso of the subject, positioned on the torso of the subject by a positioning element, which may include a belt, for example. Posture of the subject can be determined at least in part from a height measurement. In some aspects posture may be determined in part from additional measurements, e.g., inclination or force. For example, the torso support can be a back brace for providing support to the back of a subject to limit or prevent injury or discomfort.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 U.S.C §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

None.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s)from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

To the extent that the listings of applications provided above may be inconsistent with the listings provided via an ADS, it is the intent of the Application to claim priority to all applications listed in the Priority Applications section of either document.

All subject matter of the Priority Applications and the Related Applications and of any and all parent, grandparent, great-grandparent, etc. applications of the

Priority Applications and the Related Applications, including any priority claims, is incorporated herein by reference to the extent such subject matter is not inconsistent herewith.

### SUMMARY

In one aspect, an active torso support includes, but is not limited to: at least one force applying element adapted to apply force to a portion of a torso of a subject; at least one positioning element adapted to position the at least one force applying element with respect to the torso of the subject; and control circuitry including:

posture determination circuitry configured to generate a signal indicative of a posture of the subject based at least in part on a time history of the height of a portion of the active torso support; and actuation circuitry configured to control actuation of the at least one force applying element responsive to the signal indicative of the posture of the subject. In an aspect, the active torso support includes a height sensor adapted to generate a height signal indicative of the height of a portion of the active torso support, and a memory adapted to store the time history of the height signal indicative of the height of the portion of the active torso support. In another aspect, active torso support is operably coupled to a remote device that includes a height sensor adapted to generate a height signal indicative of the height of a portion of the active torso support. In another aspect, the active torso support includes an inclinometer adapted to generate an inclination signal indicative of an inclination of at least a portion of the active torso support. In addition to the foregoing, other device aspects are described in the claims, drawings, and text forming a part of the disclosure set forth herein.

In one aspect, a method of controlling an active torso support includes, but is not limited to, receiving a signal indicative of a height of a portion of an active torso support worn by a subject; determining a posture of the subject based at least in part on a time history of the height of the portion of the active torso support worn by the subject; and controlling actuation of the active torso support based on the posture of the subject. The method may include receiving a signal indicative of a height of a portion of an active torso support worn by a subject from a sensor located on the active torso support, or receiving a signal indicative of a height of a portion of an active torso support worn by a subject from a remote device located remote from the active torso support but operably coupled thereto. In an aspect, controlling actuation of the active torso support can include controlling actuation of at least one force applying element to apply force to a portion of the torso of the subject. In addition to the foregoing, other method aspects are described in the claims, drawings, and text forming a part of the disclosure set forth herein.

In one aspect, an article of manufacture includes one or more non-transitory machine-readable data storage media bearing one or more instructions for: receiving a signal indicative of a height of a portion of an active torso support worn by a subject; determining a posture of the subject based at least in part on a time history of the height of the portion of the active torso support worn by the subject; and controlling actuation of the active torso support based on the posture of the subject. In addition to the foregoing, other aspects of articles of manufacture including one or more non-transitory machine-readable data storage media bearing one or more instructions are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

For a more complete understanding of embodiments, reference now is made to the following descriptions taken in connection with the accompanying drawings.

The use of the same symbols in different drawings typically indicates similar or identical items, unless context dictates otherwise. The illustrative embodiments described in the detailed description, drawings, and claims are not meant to be limiting. Other embodiments may be utilized, and other changes may be made, without departing from the spirit or scope of the subject matter presented here.

FIG. 1 is an illustration of an active torso support.

FIG. 2 is an illustration of an active torso support.

FIG. 3 is a block diagram of an active torso support.

FIG. 4A depicts a standing subject wearing an active torso support.

FIG. 4B depicts a sitting subject wearing an active torso support.

FIG. 4C depicts a lying subject wearing an active torso support.

FIG. 4D illustrates height signal corresponding to different postures of a subject.

FIG. 5A is an illustration of a torso support including sensors for detecting height and inclination.

FIG. 5B is an illustration of a torso support including sensors for detecting height and inclination.

FIG. 5C is an illustration of a torso support including sensors for generating signals indicative of a sitting posture of a subject.

FIG. 5D is an illustration of a torso support including sensors for generating signals indicative of lying posture of a subject.

FIG. 6 is a flow diagram of a method of controlling an active torso support.

FIG. 7 is a flow diagram of a method of controlling an active torso support.

FIG. 8 is a flow diagram of a method of controlling an active torso support.

FIG. 9 illustrates an article of manufacture including non-transitory machine-readable data storage media bearing one or more instructions.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

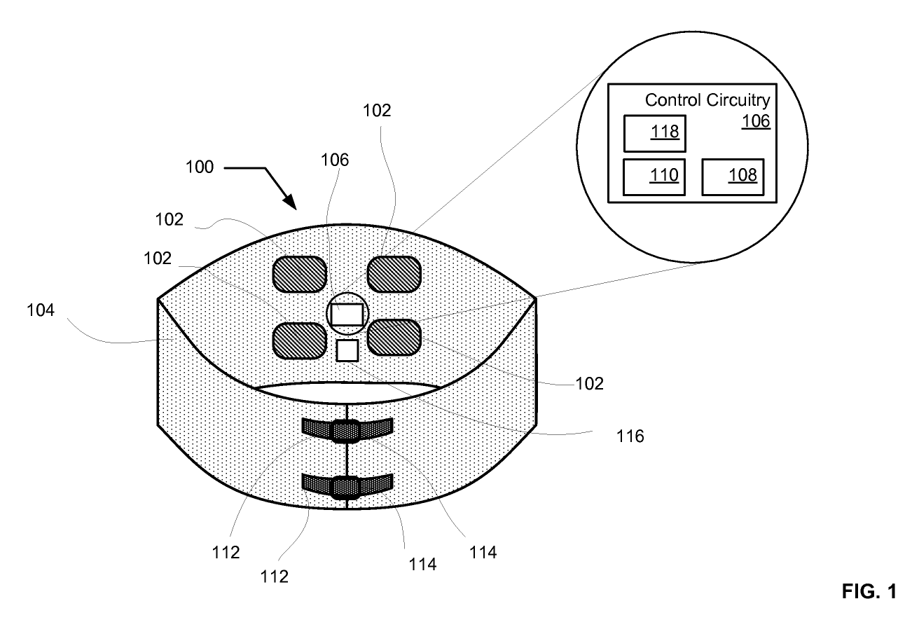

FIG. 1 depicts an active torso support 100, which includes: at least one force applying element 102 adapted to apply force to a portion of a torso of a subject; at least one positioning element 104 adapted to position the at least one force applying element with respect to the torso of the subject; and control circuitry 106 including: posture determination circuitry 108 configured to generate a signal indicative of a posture of the subject based at least in part on a time history of the height of a portion of the active torso support; and actuation circuitry 110 configured to control actuation of the at least one force applying element responsive to the signal indicative of the posture of the subject.

Active torso support 100 may include one or multiple force applying elements 102 that are capable of applying force or pressure to a region of the torso of the subject, for example, for the purpose of providing support to weak or injured muscles and/or to prevent or minimize discomfort or injury to muscles or other structures in the torso due to loading. The active torso support may be configured as a back support or back brace, as depicted in FIG. 1, but is not limited thereto, and may be configured to support or brace other portions of the torso, including, for example, one or more of all or portions of a side, an abdomen, a chest, a ribcage, a stomach, a hip, a pelvic region, an abdomen, a thorax, a shoulder, a buttock, a lower back, and an upper back.

It is contemplated that an active torso support as described herein functions generally as follows: if a particular posture or change in the posture of a subject is known to produce motion or loading of muscles and/or bony structures in the subject's torso that is likely to result in injury or discomfort, the active torso support will respond to detection of that posture or change in posture by applying force to one or more appropriate portions of the torso to provide support expected to prevent or minimize injury or discomfort. In some embodiments, the active torso support will respond to detection of that posture or change in posture by reducing force (e.g., by releasing an existing force applied by the torso support) so as to minimize injury or discomfort and/or to provide more freedom of action in connection with a change in posture.

A force applying element (e.g. force applying element 102 depicted in FIG. 1) can be any structure that is capable of applying force to a region of the torso of the subject, via a torso-contacting portion such as a pad or probe, and a controllable force-generating component that acts to move the torso contacting portion relative to the torso (e.g. by pressing against the torso and/or by applying shear forces to the torso, e.g. by engaging the surface of the torso by friction). A controllable force generating component can be controlled by actuation circuitry 110, e.g. via an electrical signal carried via an electrical connection or via a wireless signal such as an optical or electromagnetic signal transmitted from the actuation circuitry to the force applying element. Force applying element 102 may include one or more actuator, mechanical linkage, expandable element, inflatable element, pneumatic element, or hydraulic element, or other structures or components capable of applying force or pressure in a controlled fashion to a localized area of the torso. Expandable fluid/air filled bladders, are described, for example, in U.S. Pat. No. 4,135,503 to Romano; U.S. Pat. No. 6,540,707 to Stark et al, and U.S. Pat. No. 5,827,209 to Gross et all, each of which is incorporated herein by reference. Expansion of such bladders can be controlled through the use of a motorized pump and electrically controlled valves, with feedback provided by pressure sensors. Mechanically or pneumatically driven force applying elements can be, e.g. as described in U.S. Pat. No. 5,624,383 to Hazard et al., which is incorporated herein by reference. Pneumatic and hydraulic piston type force applying elements as described in U.S. Pat. No. 6,746,413 to Reinecke et al., which is incorporated herein by reference, and screw thread/worm gear assembly structures as described in U.S. Published Patent Application 2009/0030359 to Wikenheiser et al., which is incorporated herein by reference, may be positioned to press against the torso (delivering force substantially perpendicular to the skin surface), or positioned apply shear forces (i.e., force having a significant component parallel to the skin surface).

Although positioning element 104 is depicted as a belt in FIG. 1, the positioning element can be any structure capable of holding force applying element 102 in position with regard to at least a portion of the torso of the subject, and may include, for example, at least one band, strap, belt, or harness, or a garment such as a corset, girdle, jacket, vest, or brief. The positioning element may include one or multiple straps or other components, without limitation. The positioning element can be constructed from flexible, resilient, or elastic material, including but not limited to leather, fabric, webbing, mesh, cable, cord, flexible metals or polymers, or sections of rigid metals, polymers or other materials connected in such a manner that the sections can be movably fitted around the torso of the subject, e.g. by a hinge or other linkage or by one or more sections of flexible material. Positioning element 104 may include fasteners to secure the positioning element with respect to the torso of the subject, e.g. straps 112 and buckles 114 as depicted in FIG. 1, or other fasteners as are known in the art, including but not limited to buckles, snaps, zippers, latches, clips, ties, hook and loop fasteners, lacings, and so forth. Positioning element may include an active or passive tensioning component (for example, elastic) to provide for tightening of the positioning element about the torso of the subject to provide for a secure fit. In an embodiment, positioning element may simply include an elastic component which allows it to be slid onto the torso of the subject, without the need for fasteners.

Force applying elements and other system components described herein may be attached to the positioning element or held in place by pressure or friction, e.g. by being pressed between the torso of the subject and the positioning element.

In an aspect, active torso support 100 as depicted in FIG. 1 includes a height sensor 116 adapted to generate a height signal indicative of the height of a portion of the active torso support; and a memory 118 adapted to store the time history of the height signal indicative of the height of the portion of the active torso support. In an aspect, height sensor 116 includes an accelerometer, which may be, for example an integrating accelerometer.

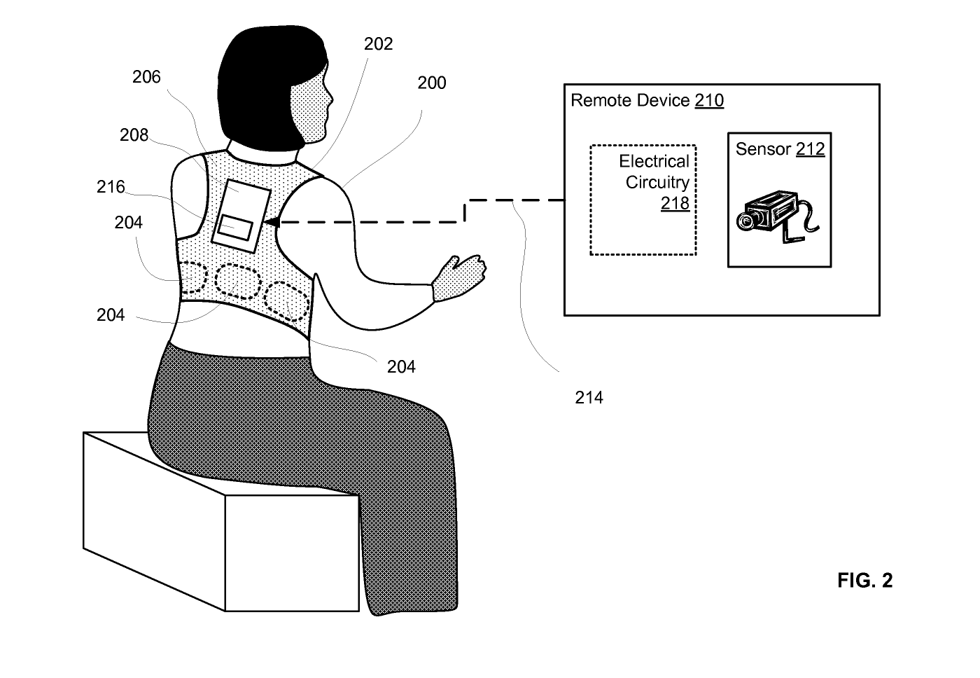

FIG. 2 depicts a subject 200 wearing an active torso support 202, which includes force applying elements 204, positioning element 206, and control circuitry 208. In an aspect, as depicted in FIG. 2, active torso support 202 is operably coupled to a remote device 210 which includes a height sensor 212 adapted to generate a height signal 214 indicative of the height of a portion of the active torso support 202. Active torso support 202 can also include a memory 216 adapted to store the time history of the height signal 214 indicative of the height of the portion of the active torso support 202. Height signal 214 from sensor 212 may be transmitted directly to active torso support 202 for processing and/or analysis by control circuitry 208 and/or storage in memory 216, or it may be subject to initial processing by electrical circuitry 218 in remote device 210. The height sensor 212 may include, for example, a video camera as depicted in FIG. 2. Electrical circuitry 218 can include suitable image processing hardware and/or software for determining height signal 214 from an image obtained from the video camera. Such image processing hardware and/or software may, for example, include or generate a model of the background of the image, segment the image, identify and the subject in the image, and analyze the image to determine parameters of the subject, such as a height parameter. Processing of an image to determine position or posture-related information may be, for example, as described in U.S. Pat. No. 7,728,839 to Yang et al., and U.S. Pat. No. 7,330,566 to Cutler et al., which are incorporated herein by reference. In particular, height and optionally width of an image or a subject can be determined as described in Cutler et al. for determining position or posture of the subject.

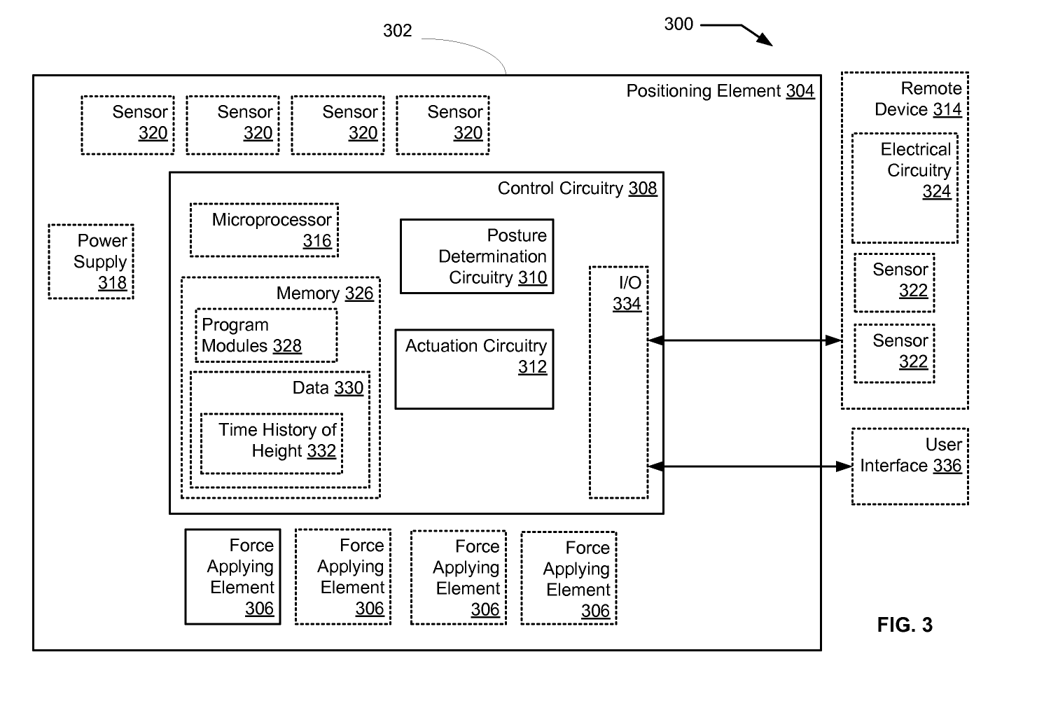

FIG. 3 is a block diagram depicting components of an active torso support system 300, including active torso support 302 including positioning element 304 and one or more force applying element 306, and control circuitry 308 including posture determination circuitry 310 and actuation circuitry 312. Optionally, torso support system 300 may include a remote device 314, e.g. as in the example depicted in FIG. 2. Control circuitry 308 may include analog or digital circuitry electrical circuitry. In an aspect, control circuitry 308 may include a microprocessor 316. Active torso support 302 may include various other elements, including power supply 318 and one or more sensors 320. Alternatively, or in addition, one or more sensors 322 may be located in remote device 314 in addition to electrical circuitry 324 for controlling operation of remote device 314. In an aspect, control circuitry 308 includes memory 326, which may store program modules 328 used in the operation of active torso support 200, and/or data 330, which may include, for example, height time history data 332. Control circuitry 308 may include I/O structure 334, which provides for communication with remote device 314, e.g. via a wired or wireless (e.g. electromagnetic or optical) connection, or with a user interface 336. Electrical circuitry 324 in remote device 314 includes any electrical circuitry needed for processing signal from sensors 322 and sending signals to or receiving signals from active torso support 302 via I/O structure 334.

In a general sense, those skilled in the art will recognize that the various embodiments described herein can be implemented, individually and/or collectively, by various types of electrical circuitry having a wide range of electrical components such as hardware, software, firmware, and/or virtually any combination thereof, limited to patentable subject matter under 35 U.S.C. §101. Electrical circuitry (including control circuitry 308 and electrical circuitry 324 depicted in FIG. 3) includes electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs (e.g., graphene based circuitry). In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

Those skilled in the art will recognize that at least a portion of the devices and/or processes described herein can be integrated into a data processing system. Those having skill in the art will recognize that a data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Force applying elements 306 are as described in connection with FIG. 1, and are typically electromechanical in nature. It will be appreciated that a wide range of components may impart mechanical force or motion, such as rigid bodies, spring or torsional bodies, hydraulics, electro-magnetically actuated devices, and/or virtually any combination thereof. As used herein “electro-mechanical system” includes, but is not limited to, electrical circuitry operably coupled with a transducer (e.g., an actuator, a motor, a piezoelectric crystal, a Micro Electro Mechanical System (MEMS), etc.). Those skilled in the art will recognize that electro-mechanical as used herein is not necessarily limited to a system that has both electrical and mechanical actuation except as context may dictate otherwise.

In an aspect, actuation circuitry 312 is configured to control actuation of the at least one force applying element 306 responsive to a change in the posture of the subject. Controlling actuation can include increasing or decreasing the force applied by the at least one force applying element 306. Actuation circuitry 312 may be configured to control actuation of the at least one force applying element 306 as a function of a rate of the change in the posture of the subject with respect to time. For example, actuation circuitry can be configured to control actuation of the at least one force applying element by actuating the at least one force applying element 306 if the change in the posture of the subject with respect to time is greater than a threshold rate of change, and/or if the change in the posture of the subject with respect to time is less than a threshold rate of change.

In an aspect, actuation circuitry 312 is configured to control actuation of the at least one force applying element 306 to provide additional support to the torso of the subject responsive to detection of the change in the posture of the subject. A change in the posture may be, for example, a change in the posture of the subject from standing to sitting, from sitting to standing, from standing to lying, from lying to standing, or various other posture changes. A change in posture may include leaning forward (from an upright sitting or standing posture, for example), or twisting the torso relative to the hips.

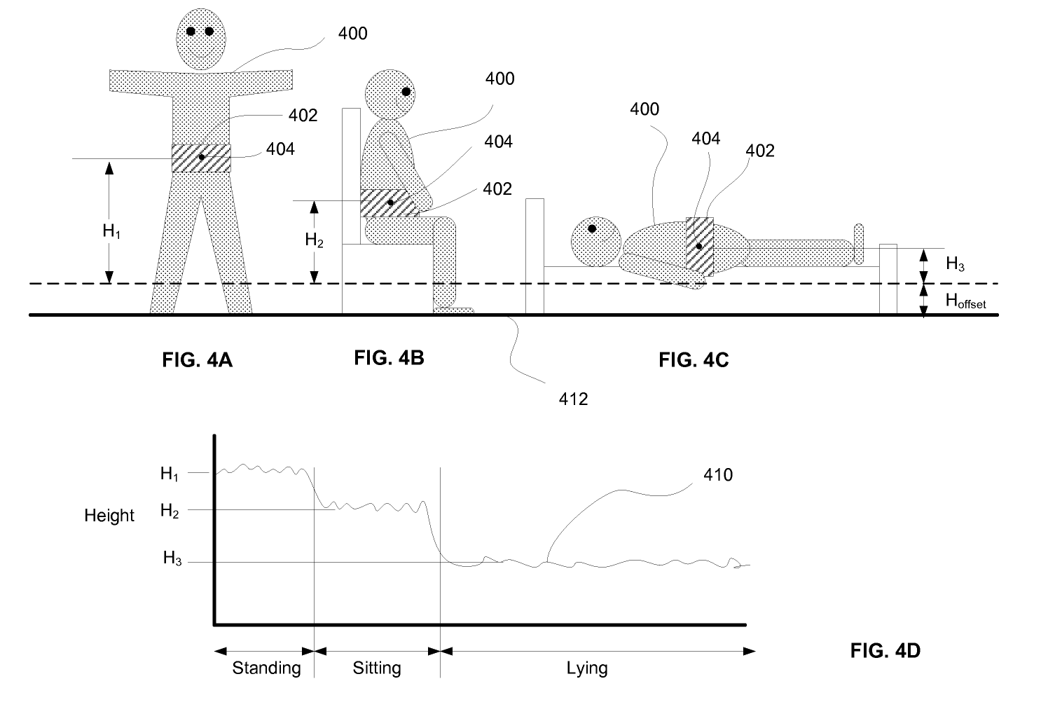

FIGS. 4A-4D illustrate detection of differences in the posture through determination of the height of a portion of a torso support. FIGS. 4A-4C depict a subject 400 wearing an active torso support 402 which includes a height sensor 404. Height sensor 404 may be, for example, an integrating accelerometer. Height sensor 404 can be configured to detect the height of the portion of the torso support at which height sensor 404 is located. In FIGS. 4A, 4B, 4C, subject 400 is in standing, sitting, and lying postures, respectively. Height sensor 404 produces a height signal 410 (a prophetic example of which is depicted generally in FIG. 4D), having different values H₁, H₂, and H₃ corresponding to standing, sitting, and lying, respectively. A height measurement obtained with an integrating accelerometer based on a time history of the height signal may be a relative height measurement. That is, height signal 410 may correctly reflect the differences in height between the standing height H₁, sitting height H₂, and lying height H₃ but not the absolute distance between, for example, the floor 412 or any other fixed reference and height sensor 404. As depicted in FIGS. 4A-4D, the absolute height of sensor 404 may not be known (that is, there may exist a constant, but unknown difference H_{offset} between the measured height and the actual height of height sensor 404 with respect to floor 412 which does not, however limit the ability to detect posture based on height). In some embodiments, however, a height sensor may produce an absolute height measure with regard to floor 412 or another reference. In some aspects, height signal 410 by itself may be sufficient to distinguish between lying, standing, and sitting. It is contemplated that in some aspects initial calibration of the system may be performed to establish absolute or relative height measurements corresponding to the various postures.

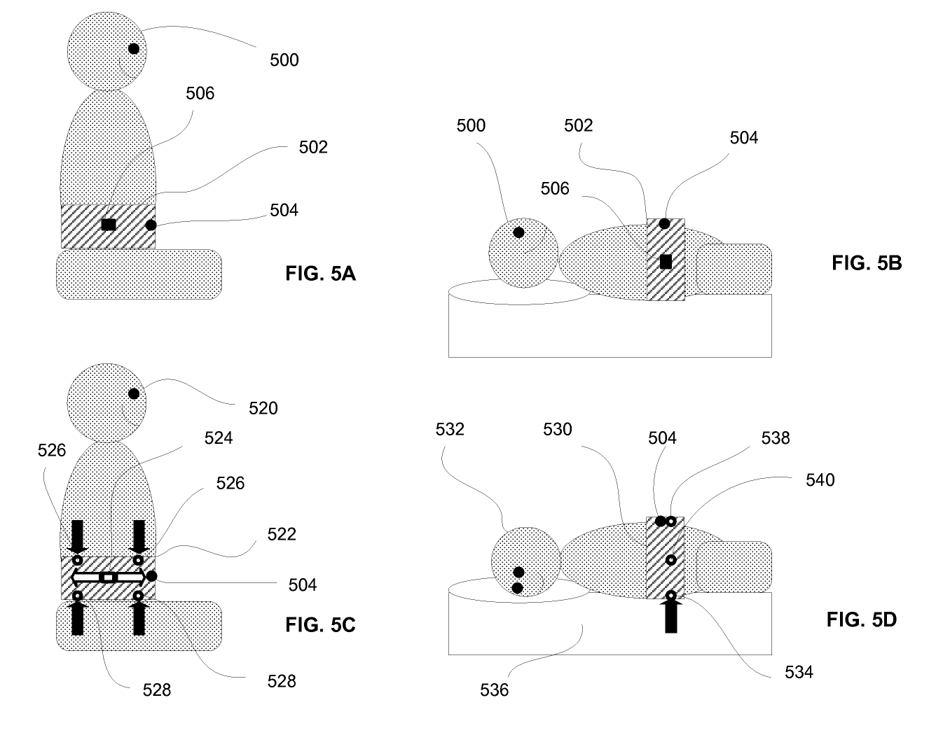

In some situations, a height measurement may not provide sufficient information to distinguish between different postures. For example, if the height of the sensor is substantially the same when the subject is sitting as when the subject is lying, then a height sensor by itself may be insufficient to distinguish between the two. However, additional sensors may be used to permit sitting to be distinguished from lying under various circumstances, and to detect other postures and/or changes in posture, as will be discussed in connection with FIGS. 5A-5D. FIG. 5A depicts a subject 500 wearing an active torso support 502 that includes a height sensor 504 and inclinometer 506. Inclinometer 506 may be adapted to generate an inclination signal indicative of an inclination or an angular orientation of at least a portion of the active torso support, and may be, for example an integrated circuit based and/or MEMS inclinometer. Even if the height of height sensor 504 is the same when subject 500 is in a sitting posture as depicted in FIG. 5A and a lying posture as depicted in FIG. 5B, inclinometer 506 is oriented to generate a different signal when subject 500 is in the sitting posture depicted in FIG. 5A and the lying posture as depicted in FIG. 5B. Signals from height sensor 504 and inclinometer 506 are provided to posture determination circuitry (not depicted in FIG. 5, but generally as described in connection with FIG. 3). Posture determination circuitry (e.g. posture determination circuitry 310 in FIG. 3, which can receive signals from multiple sensors 320) can be configured to generate a signal indicative of a posture of the subject based at least in part on the inclination signal, and actuation circuitry (e.g. actuation circuitry 312 in FIG. 3) may be configured to control actuation of the at least one force applying element (e.g. force applying element 306) responsive to the signal indicative of the posture of the subject based at least in part on the inclination signal. In an aspect, posture determination circuitry is configured to detect a change in the posture of the subject (e.g., change in posture with respect to time, as determined by evaluating the time history of the height or other signal indicating posture). Actuation circuitry may be configured to control actuation of the active torso support to provide additional support to the torso of the subject responsive to detection of the change in the posture of the subject, e.g. by actuating one or more force applying elements depending upon the posture of the subject. Posture determination circuitry may be configured to detect a change in the posture of the subject from lying to sitting, from sitting to lying, from lying to standing, from standing to lying, etc. Posture determination may be configured to detect a change in posture that includes leaning forward from an upright posture or twisting the torso relative to the hips.

Posture determination circuitry can configured to detect a change in the posture of the subject from lying on a side to lying on a back, from lying on a side to lying on a front, from lying on a back to lying on a side, or from lying on a front to lying on a side. Information regarding such posture changes may be obtained, for example, from data from one or more inclinometers, accelerometers, and/or from additional sensors on or remote from the torso support. FIGS. 5C and 5D depict examples of other possible sensor configurations. In an aspect, the active torso support may include at least one force sensor or pressure sensor adapted to generate a signal indicative of a sitting posture of the subject. For example, in FIG. 5C, subject 520 wears torso support 522 which in addition to height sensor 504 includes sensor 524, which is configured to sense tensile forces (indicated by white arrows) in the torso support 522 due to expansion of the portion of the subject's torso enclosed by torso support 522 when the subject is in a sitting posture, and sensors 526 which are configured to sense compressive forces on the upper edge of the torso support 522 and sensors 528 configured to sense compressive forces on the lower edges of the torso support 522. Compressive forces are indicated by black arrows. Alternatively, or in addition, the active torso support 530 worn by a subject 532 may include at least one force sensor or pressure sensor adapted to generate a force or pressure signal indicative of a lying posture of the subject. For example, as depicted in FIG. 5D, in addition to height sensor 504, at least one force sensor or pressure sensor 534 may be positioned on the active torso support 530 to generate a force or pressure signal indicative of a front lying posture of the subject. For example, sensor 534 may be located on the front exterior portion of the torso support 530 to detect pressure of the torso support 530 against a supporting surface such as a bed 536, as depicted, or it may be located in a front interior portion of the torso support to detect pressure of the subject's torso against the torso support. Similarly, in another aspect, at least one force sensor or pressure sensor may be positioned on the active torso support to generate a force or pressure signal indicative of a back lying posture of the subject (see sensor 538 in FIG. 5D), or to generate a force or pressure signal indicative of a side lying posture of the subject (see sensor 540 in FIG. 5D).

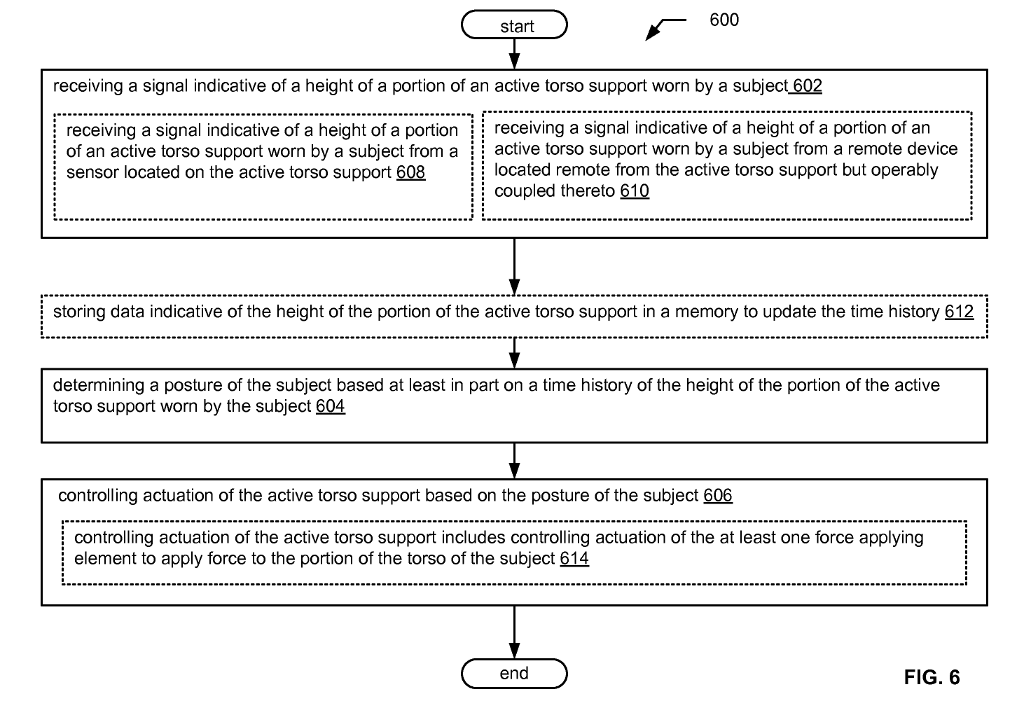

FIG. 6 is a flow diagram of a method 600 of controlling an active torso support, comprising: receiving a signal indicative of a height of a portion of an active torso support worn by a subject at 602; determining a posture of the subject based at least in part on a time history of the height of the portion of the active torso support worn by the subject at 604; and controlling actuation of the active torso support based on the posture of the subject at 606. The method may include receiving a signal indicative of a height of a portion of an active torso support worn by a subject from a sensor located on the active torso support, as indicated at 608, or receiving a signal indicative of a height of a portion of an active torso support worn by a subject from a remote device located remote from the active torso support but operably coupled thereto, as indicated at 610). Method 600 may also include storing data indicative of the height of the portion of the active torso support in a memory to update the time history, as indicated at 612. As described herein above, the active torso support can include at least one force applying element adapted to apply force to a portion of a torso of a subject and at least one positioning element adapted to position the at least one force applying element with respect to the torso of the subject, and controlling actuation of the active torso support can controlling actuation of the at least one force applying element to apply force to the portion of the torso of the subject, as indicated at 614. Controlling actuation of the active torso support based on the posture of the subject can include increasing or decreasing the force applied by the at least one force applying element. Here and elsewhere, dotted lines around a box indicate an optional or alternative part of the method.

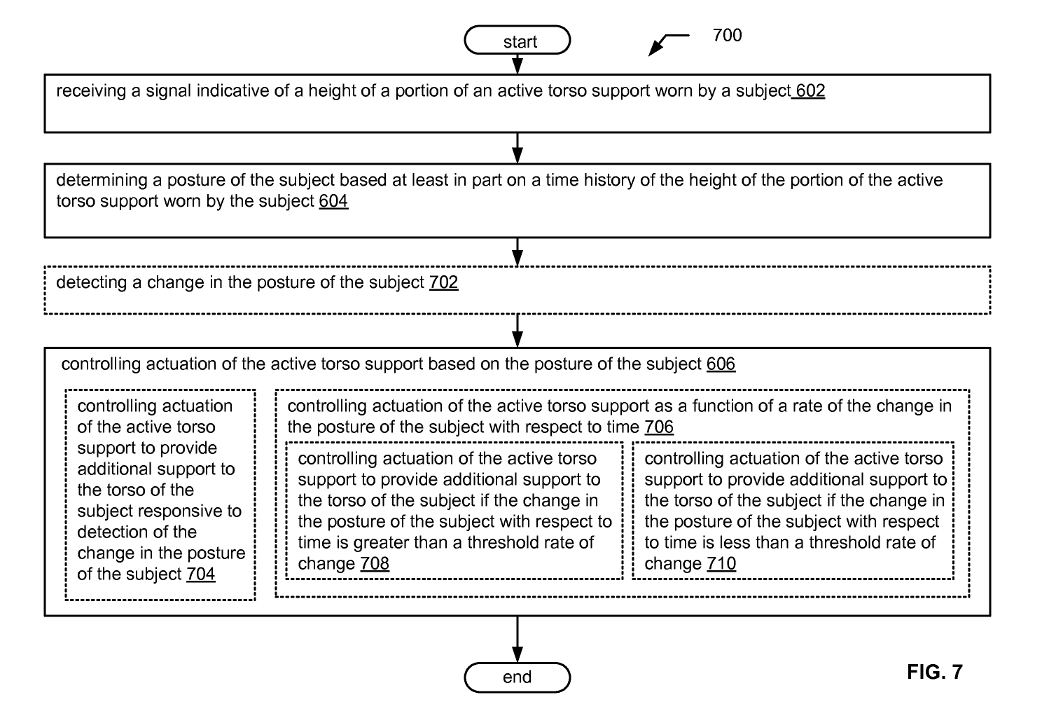

As depicted in FIG. 7, in an aspect, method 700, which is a variant of the method shown in FIG. 6, includes detecting a change in the posture of the subject, at 702. Controlling actuation of the active torso support may include controlling actuation of the active torso support to provide additional support to the torso of the subject responsive to detection of the change in the posture of the subject, as shown at 704. In an aspect, controlling actuation of the active torso support includes controlling actuation of the active torso support as a function of a rate of the change in the posture of the subject with respect to time as shown at 706, for example, controlling actuation of the active torso support to provide additional support to the torso of the subject if the change in the posture of the subject with respect to time is greater than a threshold rate of change as indicated at 708, or controlling actuation of the active torso support to provide additional support to the torso of the subject if the change in the posture of the subject with respect to time is less than a threshold rate of change as indicated at 710, or both. Detecting a change in the posture of the subject may include detecting a change from standing to sitting, from sitting to standing, from standing to lying, from lying to standing, or from upright to leaning forward, or detecting of the torso relative to the hips, as described herein above.

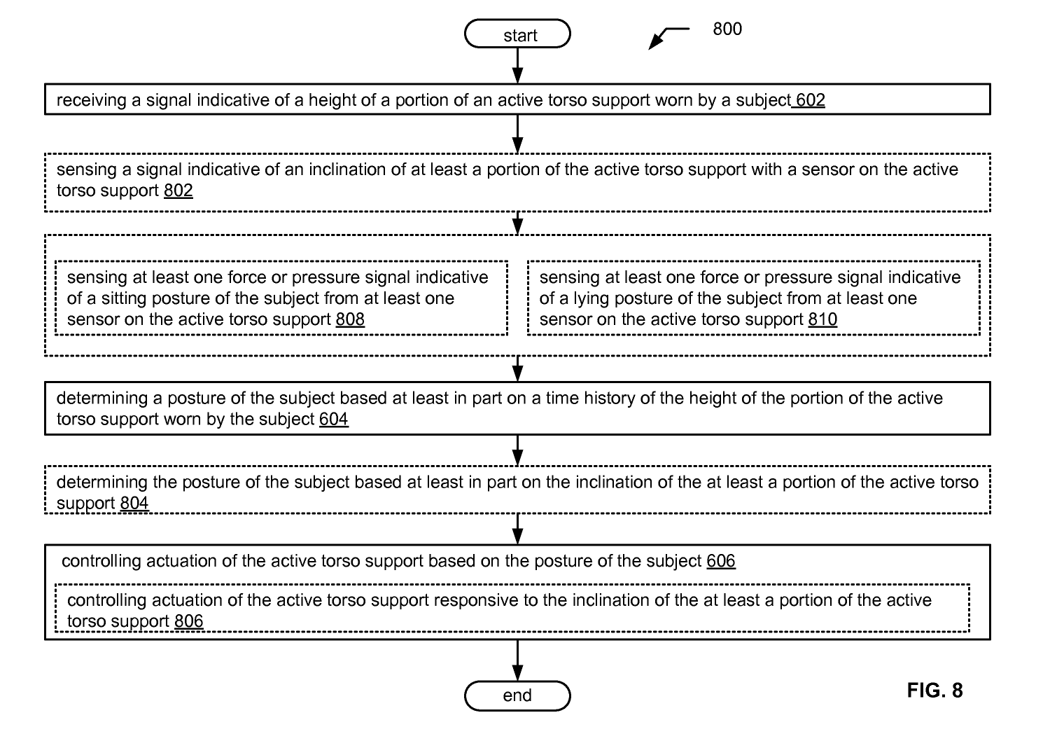

As shown in FIG. 8, in an aspect, a method 800, which is a variant of the method shown in FIG. 6, includes sensing a signal indicative of an inclination of at least a portion of the active torso support with a sensor on the active torso support, at 802. Method 800 may then include determining the posture of the subject based at least in part on the inclination of the at least a portion of the active torso support, as indicated at 804. In addition, controlling actuation of the active torso support can include controlling actuation of the active torso support responsive to the inclination of the at least a portion of the active torso support, as indicated at 806. In an aspect, method 800 includes detecting a change in the posture of the subject. In an aspect, controlling actuation of the active torso support includes controlling actuation of the active torso support to provide additional support to the torso of the subject responsive to detection of the change in the posture of the subject. For example, detecting a change in the posture of the subject can include detecting a change from lying to sitting, from sitting to lying, from upright to leaning forward, from lying on a side to lying on a back, from lying on a side to lying on a front, from lying on a back to lying on a side, or from lying on a front to lying on a side, or detecting a change that includes leaning forward or twisting the torso relative to the hips.

In an aspect, method 800 includes sensing at least one force or pressure signal indicative of a sitting posture of the subject from at least one sensor on the active torso support, as indicated at 808. In an aspect, method 808 includes sensing at least one force or pressure signal indicative of a lying posture of the subject from at least one sensor on the active torso support as indicated at 810, e.g., detecting a front lying posture of the subject based at least in part on the at least one force or pressure signal, detecting a back lying posture of the subject based at least in part on the at least one force or pressure signal, or detecting a side lying posture of the subject based at least in part on the at least one force or pressure signal.

In various embodiments, methods as described herein may be performed according to instructions implementable in either hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture, limited to patentable subject matter under 35 U.S.C. §101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware. In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled//implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof, limited to patentable subject matter under 35 U.S.C. §101. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, limited to patentable subject matter under 35 U.S.C. §101, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc.) and so forth).

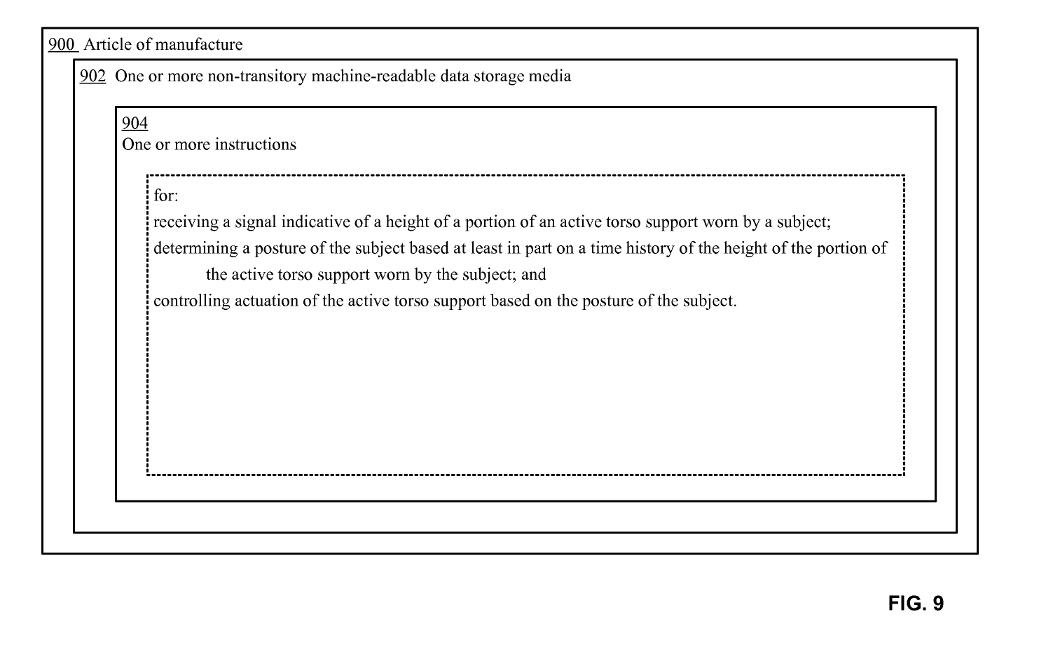

FIG. 9 depicts an article of manufacture 900 that includes one or more non-transitory machine-readable data storage media 902 bearing one or more instructions 904 for: receiving a signal indicative of a height of a portion of an active torso support worn by a subject; determining a posture of the subject based at least in part on a time history of the height of the portion of the active torso support worn by the subject; and controlling actuation of the active torso support based on the posture of the subject. Instructions 904 depicted in FIG. 9 correspond to the method 600 shown in FIG. 6. Other variants of methods as depicted in FIGS. 6-8 and as described herein can be implemented through the use of non-transitory machine-readable data storage media bearing one or more suitable instructions.

For example, the one or more non-transitory machine-readable data storage media 902 can bear one or more instructions 904 for receiving a signal indicative of a height of a portion of an active torso support worn by a subject from a sensor located on the active torso support, or one or more instructions for receiving a signal indicative of a height of a portion of an active torso support worn by a subject from a remote device located remote from the active torso support but operably coupled thereto. In an aspect, the one or more instruction for controlling actuation of the active torso support include one or more instructions for controlling actuation of at least one force applying element to apply force to the portion of the torso of the subject, or decreasing a force applied to the torso of the subject. In an aspect, the one or more non-transitory machine-readable data storage media 902 bear one or more instructions for storing data indicative of the height of the portion of the active torso support in a memory to update the time history.

In an aspect, the one or more non-transitory machine-readable data storage media 902 bear one or more instructions 904 for detecting a change in the posture of the subject. The one or more non-transitory machine-readable data storage media 902 may bear one or more instructions for controlling actuation of the active torso support to provide additional support to the torso of the subject responsive to detection of the change in the posture of the subject. The one or more non-transitory machine-readable data storage media 902 may bear one or more instructions 904 for controlling actuation of the active torso support as a function of a rate of the change in the posture of the subject with respect to time, for example, one or more instructions for controlling actuation of the active torso support to provide additional support to the torso of the subject if the change in the posture of the subject with respect to time is greater than a threshold rate of change, and/or controlling actuation of the active torso support to provide additional support to the torso of the subject if the change in the posture of the subject with respect to time is less than a threshold rate of change. The one or more instructions for detecting a change in the posture of the subject may include one or more instructions for detecting a change from standing to sitting, detecting a change from sitting to standing, detecting a change from standing to lying, or detecting a change from lying to standing, detecting a change from upright to leaning forward, or detecting twisting or the torso relative to the hips, for example.

In an aspect, non-transitory machine-readable data storage media 902 bear one or more instructions 904 for sensing a signal indicative of an inclination of at least a portion of the active torso support with a sensor on the active torso support. The non-transitory machine-readable data storage media 902 may then also bear one or more instructions for determining the posture of the subject based at least in part on the inclination of the at least a portion of the active torso support, and/or for controlling actuation of the active torso support responsive to the inclination of the at least a portion of the active torso support. The non-transitory machine-readable data storage media 902 may bear one or more instructions for detecting a change in the posture of the subject. The one or more instructions for controlling actuation of the active torso support include one or more instructions for controlling actuation of the active torso support to provide additional support to the torso of the subject responsive to detection of the change in the posture of the subject. As described above, instructions for detecting a change in the posture of the subject may include one or more instructions for detecting a change from lying to sitting, from sitting to lying, from lying on a side to lying on a back, from lying on a side to lying on a front, from lying on a back to lying on a side, from lying on a front to lying on a side, from upright to leaning forward, or twisting the torso relative to the hips.

In an aspect, the one or more non-transitory machine-readable data storage media 902 bear one or more instructions 904 for sensing at least one force or pressure signal indicative of a sitting posture of the subject from at least one sensor on the active torso support. In an aspect, the one or more non-transitory machine-readable data storage media 902 bear one or more instructions 904 for sensing at least one force or pressure signal indicative of a lying posture of the subject from at least one sensor on the active torso support, including, for example detecting a front lying posture, a back lying posture, or a side lying posture of the subject, based at least in part on the at least one force or pressure signal.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An active torso support, comprising:
at least one force applying element adapted to apply force to a portion of a torso of a subject;
at least one positioning element adapted to position the at least one force applying element with respect to the torso of the subject; and
control circuitry including:
posture determination circuitry configured to generate a signal indicative of a posture of the subject based at least in part on a time history of the height of a portion of the active torso support; and
actuation circuitry configured to control actuation of the at least one force applying element responsive to the signal indicative of the posture of the subject.

2. (canceled)

3. (canceled)

4. The active torso support of claim 1, comprising:
a height sensor adapted to generate a height signal indicative of the height of a portion of the active torso support; and
a memory adapted to store the time history of the height signal indicative of the height of the portion of the active torso support.

5. The active torso support of claim 1, wherein the active torso support is operably coupled to a remote device including a height sensor adapted to generate a height signal indicative of the height of a portion of the active torso support.

6. The active torso support of claim 5, comprising a memory adapted to store the time history of the height signal indicative of the height of the portion of the active torso support.

7. The active torso support of claim 4, wherein the height sensor includes an accelerometer.

8. (canceled)

9. The active torso support of claim 1, wherein the actuation circuitry is configured to control actuation of the at least one force applying element responsive to a change in the posture of the subject.

10. The active torso support of claim 9, wherein the actuation circuitry is configured to control actuation of the at least one force applying element as a function of a rate of the change in the posture of the subject with respect to time.

11.-19. (canceled)

20. The active torso support of claim 1, comprising an inclinometer adapted to generate an inclination signal indicative of an inclination of at least a portion of the active torso support.

21. The active torso support of claim 20, wherein the posture determination circuitry is configured to generate a signal indicative of the posture of the subject based at least in part on the inclination signal.

22. (canceled)

23. The active torso support of claim 21, wherein the posture determination circuitry is configured to detect a change in the posture of the subject.

24. The active torso support of claim 23, wherein the actuation circuitry is configured to control actuation of the active torso support to provide additional support to the torso of the subject responsive to detection of the change in the posture of the subject.

25.-38. (canceled)

39. The active torso support of claim 1, wherein the force applying element includes at least one actuator, mechanical linkage, expandable element, inflatable element, pneumatic element, or hydraulic element.

40. The active torso support of claim 1, wherein the positioning element includes at least one band, strap, belt, or garment.

41. A method of controlling an active torso support, comprising:
receiving a signal indicative of a height of a portion of an active torso support worn by a subject;
determining a posture of the subject based at least in part on a time history of the height of the portion of the active torso support worn by the subject; and
controlling actuation of the active torso support based on the posture of the subject.

42. The method of claim 41, wherein the active torso support includes at least one force applying element adapted to apply force to a portion of a torso of a subject and at least one positioning element adapted to position the at least one force applying element with respect to the torso of the subject, and wherein controlling actuation of the active torso support includes controlling actuation of the at least one force applying element to apply force to the portion of the torso of the subject.

43.-44. (canceled)

45. The method of claim 41, comprising receiving a signal indicative of a height of a portion of an active torso support worn by a subject from a sensor located on the active torso support.

46. The method of claim 41, comprising receiving a signal indicative of a height of a portion of an active torso support worn by a subject from a remote device located remote from the active torso support but operably coupled thereto.

47. The method of claim 41, comprising storing data indicative of the height of the portion of the active torso support in a memory to update the time history.

48. The method of claim 41, comprising detecting a change in the posture of the subject.

49. (canceled)

50. The method of claim 48, wherein controlling actuation of the active torso support includes controlling actuation of the active torso support as a function of a rate of the change in the posture of the subject with respect to time.

51.-58. (canceled)

59. The method of claim 41, comprising sensing a signal indicative of an inclination of at least a portion of the active torso support with a sensor on the active torso support.

60. The method of claim 59, comprising determining the posture of the subject based at least in part on the inclination of the at least a portion of the active torso support.

61. (canceled)

62. The method of claim 60, comprising detecting a change in the posture of the subject.

63.-75. (canceled)

76. An article of manufacture comprising:
one or more non-transitory machine-readable data storage media bearing one or more instructions for:
receiving a signal indicative of a height of a portion of an active torso support worn by a subject;
determining a posture of the subject based at least in part on a time history of the height of the portion of the active torso support worn by the subject; and
controlling actuation of the active torso support based on the posture of the subject.

77.-109. (canceled)

110. The active torso support of claim 9, wherein the posture determination circuitry is configured to detect at least one of a change in the posture of the subject from standing to sitting, a change in the posture of the subject from sitting to standing, a change in the posture of the subject from standing to lying, a change in the posture of the subject from lying to standing, a change in the posture of the subject from upright to leaning forward, and a change in the posture of the subject including twisting of the torso relative to the hips.

111. The active torso support of claim 23, wherein the posture determination circuitry is configured to detect at least one of a change in the posture of the subject from lying to sitting, a change in the posture of the subject from sitting to lying, a change in the posture of the subject from lying to standing, a change in the posture of the subject from standing to lying, a change in the posture of the subject from upright to leaning forward, a change in the posture of the subject from lying on a side to lying on a back, a change in the posture of the subject from lying on a side to lying on a front, a change in the posture of the subject from lying on a back to lying on a side, and a change in the posture of the subject from lying on a front to lying on a side.

112. The active torso support of claim 1, comprising at least one force sensor or pressure sensor adapted to generate a signal indicative of a sitting posture of the subject or a lying posture of the subject.

113. The active torso support of claim 112, wherein the at least one force sensor or pressure sensor is positioned on the active torso support to generate a force or pressure signal indicative of a posture selected from the group consisting of a front lying posture of the subject, a back lying posture of the subject, and a side lying posture of the subject.

114. The method of claim 42, wherein controlling actuation of the active torso support based on the posture of the subject includes at least one of increasing the force applied by at least one force applying element and decreasing the force applied by at least one force applying element.

115. The method of claim 48, wherein detecting a change in the posture of the subject includes at least one of detecting a change from standing to sitting, detecting a change from sitting to standing, detecting a change from standing to lying, detecting a change from lying to standing, detecting a change from upright to leaning forward, and detecting twisting of the torso relative to the hips.

116. The method of claim 62, wherein detecting a change in the posture of the subject includes at least one of detecting a change from lying to sitting, detecting a change from sitting to lying, detecting a change from upright to leaning forward, detecting a change from lying on a side to lying on a back, detecting a change from lying on a side to lying on a front, detecting a change from lying on a back to lying on a side, and detecting a change from lying on a front to lying on a side.

117. The method of claim 41, comprising at least one of sensing at least one force or pressure signal indicative of a sitting posture of the subject from at least one sensor on the active torso support and sensing at least one force or pressure signal indicative of a lying posture of the subject from at least one sensor on the active torso support.

118. The method of claim 117, comprising detecting at least one of a front lying posture of the subject, a back lying posture of the subject, and a side lying posture of the subject based at least in part on the at least one force or pressure signal indicative of a lying posture of the subject.
